(19) United States
(12) Patent Application Publication
(10) Pub. No.: US 2026/0192022 A1
(43) Pub. Date: Jul. 9, 2026

(54) Biodegradable Splint And Method Of Producing The Same

(71) Applicant: Stryker European Operations Limited, Carrigtwohill, Co Cork (IE)

(72) Inventor: Johannes Jacobus Jacobs, Dublin (IE)

(73) Assignee: Stryker European Operations Limited, Carrigtwohill, Co Cork (IE)

(21) Appl. No.: 19/133,398

(22) PCT Filed: Nov. 27, 2023
(86) PCT No.: PCT/IB2023/061939

§ 371 Date: May 28, 2025

Related U.S. Application Data
(60) Provisional application No. 63/385,075, filed on Nov. 28, 2022.

(51) Int. Cl. A61L 27/56; A61F 2/00; A61F 2/18; A61L 27/18; A61L 27/20; A61L 27/54

(52) U.S. Cl. CPC A61L 27/56; A61F 2/186; A61L 27/18; A61L 27/20; A61L 27/54; A61F 2002/0081; A61F 2210/0004; A61F 2250/0067; A61L 2400/04

(57) ABSTRACT

Biodegradable splints and methods for producing the biodegradable splints include a carrier and a foam at least partially surrounding the carrier. The carrier extends from a proximate end to a distal end and increases the structural rigidity of the biodegradable splint. The foam has a porosity of 80-99% and includes a phase-separated polymer including an amorphous segment and a crystalline segment.

### CROSS-REFERENCE TO RELATED APPLICATIONS

The subject patent application claims priority to, and all the benefits of, U.S. Provisional Patent Application No. 63/385,075, filed on Nov. 28, 2022, the entire contents of which are incorporated by reference herein.

### BACKGROUND

There is a need for bio-compatible, biodegradable synthetic devices that can be applied with improved physical and mechanical properties in an ostium of the body, such as nasal cavities. Current nasal splints may require sutures to ensure they retain their position. Removal of such devices may cause additional trauma to the patient. It is a particular objective of the present invention to overcome the drawbacks and the problems associated with the devices of the prior art.

### SUMMARY

This summary introduces a selection of concepts in a simplified form that are further described below in the Detailed Description below. This Summary is not intended to limit the scope of the claimed subject matter nor identify key features or essential features of the claimed subject matter.

In one aspect, a biodegradable splint for a nasal cavity is provided. The biodegradable splint includes a carrier extending from a proximate end to a distal end for increasing the structural rigidity of the biodegradable splint. The carrier defines a plurality of voids. A foam at least partially surrounds the carrier and is disposed in the plurality of voids. The foam has a porosity of 80-99% and includes a phase-separated polymer including an amorphous segment and a crystalline segment. The bridgeable splint also includes a tissue-adhesive polymer for covalent bonding to tissue in the nasal cavity. The tissue-adhesive polymer includes a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof.

In another aspect, a method for producing a biodegradable splint for use in a nasal cavity is provided. The method includes providing the phase-separated polymer including an amorphous segment and a crystalline segment. The method further includes providing the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The method further includes blending the phase-separated polymer with the tissue-adhesive polymer to create a blended mixture. The method further includes providing a carrier extending from a proximate end to a distal end and defining a plurality of voids. The method further includes combining the carrier and the blended mixture in a mold. Finally, the method includes forming a foam having a porosity of 80-99% from the blended mixture with the foam disposed within the plurality of voids.

In another aspect, a biodegradable splint for a nasal cavity is provided. The biodegradable splint includes the carrier extending from a proximate end to a distal end for increasing the structural rigidity of the biodegradable splint. The biodegradable splint includes the foam at least partially surrounding the carrier, the foam having a porosity of 80-99% and including a phase-separated polymer including an amorphous segment and a crystalline segment. The biodegradable splint further includes the tissue-adhesive polymer dispersed throughout the foam for covalent bonding, the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The biodegradable splint further includes a chitosan hemostatic agent and a buffer agent for establishing an environment in the nasal cavity to promote covalent bonding between the tissue-adhesive polymer and tissue in the nasal cavity.

In another aspect, a method for producing a biodegradable splint for use in a nasal cavity is provided. The method includes providing a phase-separated polymer including an amorphous segment and a crystalline segment. The method further includes providing the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The method further includes providing the chitosan hemostatic agent and providing the buffer agent for establishing an environment in the nasal cavity to promote covalent bonding between the tissue-adhesive polymer and a septum of the nasal cavity. The method further includes blending the phase-separated polymer, the chitosan hemostatic agent, the buffer agent, and the tissue-adhesive polymer to create a blended mixture. The method further includes providing the carrier extending from a proximate end to a distal end and combining the carrier and the blended mixture in a mold. The method further includes forming a foam having a porosity of 80-99% from the blended mixture to produce the biodegradable splint.

### BRIEF DESCRIPTION OF THE DRAWINGS

Advantages of the present invention will be readily appreciated as the same becomes better understood by reference to the following detailed description when considered in connection with the accompanying drawings.

FIG. 1 illustrates a biodegradable splint in a nasal cavity of a patient.

FIG. 2 illustrates a top view of one implementation of the biodegradable splint.

FIG. 3 illustrates a side view of the biodegradable splint of FIG. 2.

FIG. 4 illustrates a top view of another implementation of the biodegradable splint.

FIG. 5 illustrates another implementation of the biodegradable splint in a nasal cavity of the patient.

### DETAILED DESCRIPTION

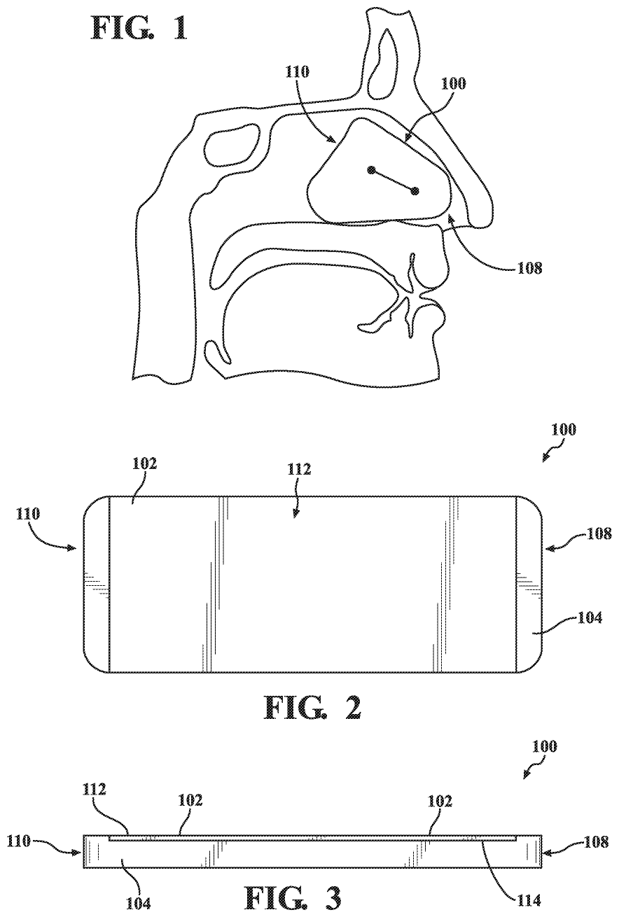

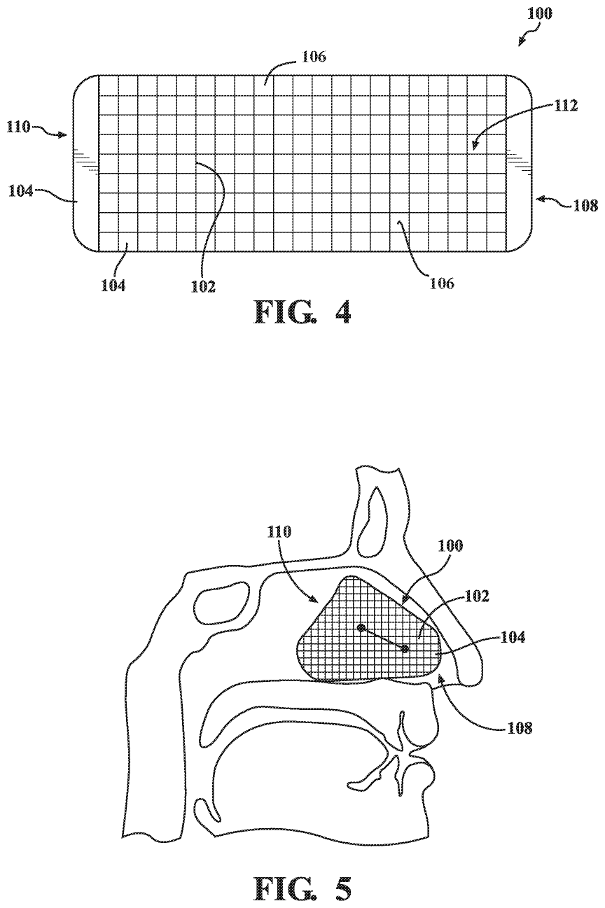

FIG. 1 illustrates one aspect of a biodegradable splint 100 for a nasal cavity. As best shown in FIGS. 2 to 4, the biodegradable splint 100 includes a carrier 102 for increasing the structural rigidity of the biodegradable splint 100. The biodegradable splint 100 also includes a foam 104. As described further below and illustrated in FIG. 4, the carrier 102 may define a plurality of voids 106. In some implementations, the foam 104 may at least partially surround the carrier 102. In other implementations, the foam 104 entirely surrounds the carrier 102, such that the carrier 102 is not visible and entirely enveloped by the foam 104. Alternatively, a portion of the carrier 104 may be visible due to the foam only partially surrounding the carrier 102. The foam 104 may be disposed in the plurality of voids 106.

The term “biodegradable” as used herein, refers to the ability of the biodegradable splint 100 to be acted upon biochemically in general by living cells or organisms or part of these systems, including hydrolysis, and to degrade and disintegrate into chemical or biochemical products. Thus, both the carrier 102 and the foam 104 of the biodegradable splint 100 are biodegradable.

The foam 104 may include a phase-separated polymer including an amorphous segment and a crystalline segment. The phase-separated polymer of the foam 104 may be selected from the list consisting of polyesters, polyhydroxyacids, polylactones, polyetheresters, polycarbonates, polydioxanes, polyanhydrides, polyurethanes, polyester(ether)urethanes, polyurethane urea, polyamides, polyesteramides, poly-orthoesters, polyaminoacids, polyphosphonates, polyphosphazenes and combinations thereof. The phase-separated polymer may also be chosen from copolymers, mixtures, composites, cross-linking and blends of the above-mentioned polymers.

The term “blend” as used herein refers to a mixture of two or more different polymers, i.e., the polymer (base polymer) and the hemostatic agent (hemostatic polymer). As used herein, the term “blend” and “polymer blend” can be used interchangeably. The mixture of polymers may be macroscopically homogeneous. The polymers in a polymer blend are preferably randomly distributed throughout the blend. In a polymer blend, cross-linking between the two or more different polymers is typically avoided. Accordingly, the degree of cross-linking of the polymers in the polymer blend is typically 0.01 or lower, more preferably 0.001 or lower, most preferably about 0. The degree of cross-linking is a well-known parameter and may also be referred to as cross-linking density. It is a measure of the amount of bondings between two polymer chains and may in particular refer to the number of bondings formed in and between the polymers of the blend per total amount of monomer units of the two or more polymers present in the blend.

The term “amorphous” as used herein, refers to segments present in the phase-separated polymer with at least one glass transition temperature below the temperature of the antrums or other cavities of the human or animal body into which the foam 104 is packed, and may also refer to a combination of an amorphous and crystalline segment which is completely amorphous when packed in the human or animal body. For example, PEG in a pre-polymer may be crystalline in pure form, but may be amorphous when comprised in the R segment of a polyurethane of the formula (I) or (II). Longer PEG segments may also be partly crystalline when comprised in the R segment of a polyurethane of the formula (I) or (II), but will become amorphous and dissolve when placed in contact with water. Therefore such longer PEG segments are part of the soft segment of the phase separated polymer of the formulas (I) or (II), whereas the hard segment should remain crystalline in nature to provide sufficient support to a foam 104 in the wet and packed state for, at least, a certain period of time.

The term “crystalline” as used herein, refers to segments, present in the phase-separated polymer, that are crystalline when packed in the human or animal body, i.e., that have a melting temperature above the temperature of the antrums or other cavities of the human or animal body into which the foam 104 is packed.

A “hydrophilic segment” as used herein, refers to a segment comprising at least one, alternatively at least two, or alternatively still at least three hydrophilic groups such as can be provided for instance by C—O—C, or ether, linkages. A hydrophilic segment may thus be provided by a polyether segment. A hydrophilic segment may also be provided by polypeptide, poly(vinyl alcohol), poly(vinylpyrrolidone) or poly(hydroxymethylmethacrylate). A hydrophilic segment is preferably derived from polyalkyleneglycol, such as polyethyleneglycol, polypropyleneglycol, or polybutyleneglycol. The preferred hydrophilic segment is a polyethyleneglycol (PEG) segment.

The term “segment” as used herein, refers to a polymeric structure of any length. In the art of polymer technology, a long polymeric structure is often referred to as a block, whereas a short polymeric structure is often referred to as a segment. Both of these conventional meanings are understood to be comprised in the term “segment” as used herein.

The phase-separated polymer includes the amorphous segment and the crystalline segment, with the amorphous segment including a hydrophilic segment. Such a polymer is described in WO-A-2004/062704, which is hereby incorporated by referenced in its entirety. These phase-separated polymers were found to show a particular good enhanced hemostatic effect in combination with hemostatic agents. This makes it possible to provide hemostatic foam 104s having very desirable mechanical, structural and chemical properties.

As described in WO-A-2004/062704, the amorphous segment of the phase-separated polymer must comprise a hydrophilic segment. This amorphous segment, also called the amorphous phase in the art, is amorphous when applied to a bleeding surface, i.e. when wet, despite the fact that it may comprise a crystalline polyether. This means that, in the dry state, the crystalline polyether may provide the amorphous phase of the polymer with partially crystalline properties. The performance of the foam 104 when applied to a bleeding surface determines the characteristics of the foam 104: when applied to a bleeding surface, the foam 104 includes an amorphous hydrophilic soft segment or phase and a crystalline hard segment or phase.

Hydrophilic groups may also be present in the hard segment of the phase-separated polymer, but the presence of hydrophilic groups in the hard segment should not result in immediate disintegration of the foam 104 when placed in contact with fluids. The crystalline hard segment or phase provides the foam 104 with rigidity, keep the foam 104 intact and prevent excessive swelling of the foam 104 when placed in contact with fluids. However, there is always some level of swelling seeing as fluid is absorbed. In particular, the crystalline linkages prevent the foam 104 from dissolving in an aqueous medium. In contrast, an organic medium, such as dioxane, would break those physical crosslinks and dissolve the entire foam and so solubilize the base polymer.

In certain implementations, the phase-separated polymer is a polymer that may comprise one or more urea, urethane, amide, carbonate, ester or anhydride link. Typically, the phase-separated polymer is a polyurea, polyamide or polyurethane, or most typically a polyurethane.

A phase-separated polyurethane is characterized by the presence of at least two immiscible or partly miscible phases with a different morphology and different thermal state at normal environmental conditions. Within one material a soft rubbery amorphous phase and a hard crystalline phase (at a temperature above the glass transition temperature of the amorphous phase and below the melting temperature of the crystalline phase) may be present or a hard glassy amorphous phase and a hard crystalline phase (at a temperature below the glass transition temperature of the amorphous phase). Also at least two amorphous phases can be present, e.g. one hard glassy and one soft rubbery phase. Even above the melting temperature, the liquid and rubbery phases can still be immiscible. More in particular, when a polyurethane has an amorphous phase and a crystalline phase, which two phases are immiscible with each other, the polyurethane is said to be phase-separated. The presence of immiscible phases (amorphous and crystalline) may be suitably determined by the use of a e.g. (modulated) differential scanning calorimetry (DSC).

The phase separated morphology provides for the mechanical properties of the foam 104. Both phases contribute to the unique properties of the material of the phase-separated polymer. The soft, amorphous phase is responsible for the flexible and elastic behavior. The hard, crystalline phase is responsible for the hardness and strength of the material. The (semi) crystalline hard segments undergo intermolecular crystallization and behave as physical cross-links and knot the soft segments in a three dimensions network structure. Because of this, microphase separation may appear where the hard crystalline and the soft amorphous segments can form a co-continuous two-phase system. Co-continuous microstructures are characterized by having both phases interpenetrating each other in three dimensions. In a co-continuous morphology all the hard areas are connected with each other. Due to this morphology the foam 104 has the ability to essentially maintain its compression strength upon blood absorption.

In one implementation of the foam 104, the phase-separated polymer is biodegradable and represented by formula (I):

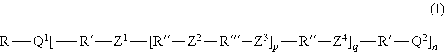

wherein R is a polymer or copolymer selected from one or more aliphatic polyesters, polyether esters, polyethers, polyanhydrides, and/or polycarbonates, and at least one R comprises a hydrophilic segment; R′, R″ and R′″ are independently C₂-C₈ alkylene, optionally substituted with C₁-C₁₀ alkyl or C₁-C₁₀ alkyl groups substituted with protected S, N, P or O moieties and/or comprising S, N, P or O in the alkylene chain; Z¹-Z⁴ are independently amide, urea or urethane, Q¹ and Q² are independently urea, urethane, amide, carbonate, ester or anhydride, n is an integer from 5-500; and p and q are independent 0 or 1.

The soft segment of the polymer of formula (I) is generally represented by R, whereas the remainder of formula (I) generally represents the hard segment of the polymer.

Although Z¹-Z⁴ may differ from each other, Z¹-Z⁴ are preferably chosen to be the same. More preferably, Z¹-Z⁴ are all urethane moieties, and the polymer can in such a case be represented by formula (II):

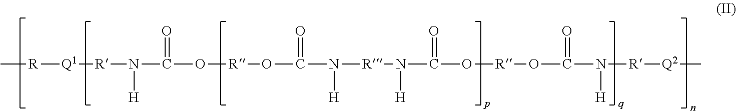

wherein Q¹, Q², R, R′, R″, R′″, p, q and n are defined as described hereinabove for formula (I).

Q¹ and Q² are chosen independently from each other from the group consisting of urea, urethane, amide, carbonate, ester and anhydride. Preferably, Q¹ and Q² are independently chosen from urethane, carbonate and ester. Although Q¹ and Q² may be chosen to be different kind of moieties, Q¹ and Q² are preferably the same, most preferably both urethane moieties.

Typically, q=1 in formulas (I) and (II). Thus, the polymer has a hard segment of sufficient length to easily form crystalline domains, resulting in a phase-separated polyurethane. An even more desirable length is obtained for this purpose if both q and p equal 1.

To enhance the phase-separated nature of the polymer, R can be chosen as a mixture of an amorphous and a crystalline segment. For this purpose, R is preferably a mixture of at least one crystalline polyester, polyether ester or polyanhydride segment and at least one amorphous aliphatic polyester, polyether, polyanhydride and/or polycarbonate segment. This may be particularly desirable when q is chosen 0, because the urethane moiety may in such a case be too small to form crystalline domains, resulting in a mixture of both phases, wherein no phase-separation occurs.

The amorphous segment may be comprised in the —R— part of the polymer according to formula a). The remaining part of the polymer according to formula (I), including the R′, R″ and R′″ units, represents the crystalline segment. The crystalline segment is always a hard segment, while the amorphous segment at least comprises one or more soft segments. R in formula (I) comprises the soft segments, while the remainder of formula 1 typically comprises the hard segments. The soft segments are typically amorphous in the phase-separated polymer. The hard segments have a tendency to crystallize, but may be amorphous when not crystallized completely.

R is a polymer or copolymer selected from aliphatic polyesters, polyether esters, polyethers, polyanhydrides, polycarbonates and combinations thereof, wherein at least one hydrophilic segment is provided in at least one amorphous segment of R. Preferably, R is a polyether ester. R can for example be a polyether ester based on DL lactide and ϵ-caprolactone, with polyethylene glycol provided in the polyether ester as a hydrophilic segment.

R comprises a hydrophilic segment and such a hydrophilic segment can very suitably be an ether segment, such as a polyether segment derivable from such polyether compounds as polyethyleneglycol, polypropyleneglycol or polybutyleneglycol. Also, a hydrophilic segment comprised in R may be derived from polypeptide, poly(vinyl alcohol), poly(vinylpyrrolidone) or poly(hydroxymethylmethacrylate). A hydrophilic segment is preferably a polyether.

Each of the groups R′, R″ and R′″ is a C₂-C₈ alkylene moiety, preferably a C₃-C₆ alkylene moiety. The alkylene moiety may be substituted with C₁-C₁₀ alkyl or C₁-C₁₀ alkyl groups substituted with protected S, N, P or O moieties and/or comprising S, N, P or O in the alkylene chain. Preferably, the alkylene moiety is unsubstituted (C₂—H₂ₙ) or substituted. R′, R″ and R′″ may all be chosen to be a different alkylene moiety, but may also be the same.

Preferably, R′ is an unsubstituted C₄ alkylene (C₄H₈) or an unsubstituted C₆ alkylene (C₆H₁₂). R′ may be derived from a diisocyanate of the formula O═C═N—R′N═C═O, such as alkanediisocyanate, preferably 1,4-butanediisocyanate (BDI) or 1,6-hexanediisocyanate (HDI).

Preferably, R″ is an unsubstituted C₄ alkylene (C₄H₈) or an unsubstituted C₃ alkylene (C₃H₆). R″ may be derived from a diol of the formula HO—R″—OH, such as 1,4-butanediol (BDO) or 1,3-propanediol (PDO).

Preferably, R′″ is an unsubstituted C₄ alkylene (C₄H₈) or an unsubstituted C₆ alkylene (C₆H₁₂). R′ may be derived from a diisocyanate of the formula O═C═N—R″—N═C═O, such as alkanediisocyanate, preferably 1,4-butanediisocyanate (BDI) or 1,6-hexanediisocyanate (HDI).

A method for preparing phase-separated biodegradable polymer of formula (I) is known in the art, such as for example described in WO-A-2004/062704.

An example of the phase separated polyurethane is provided in formula (I), wherein R is a polyether ester based on DL lactide and ϵ-caprolactone, which polyether ester comprises a hydrophilic polyethylene glycol segment; R′, R″ and R′″ are C₄ alkylene (C₄H₈); Q¹, Q² and Z¹-Z⁴ are urethane and p=1 and q=1.

Another example of the phase-separated polymer is a structure wherein R=soft segment based on DL lactide and ϵ-caprolactone and polyvinylpyrrolidone as the hydrophilic segment.

Another example of the phase-separated polymer is a structure wherein R=soft segment based on DL lactide and ϵ-caprolactone and polyvinyl alcohol as hydrophilic segment.

For these latter two structures, R′, R″ and R′″ are C₄; Q¹, Q² and Z¹-Z⁴ are urethane; and p=1 and q=1.

The foam 104 of the disclosure may be referred to as “bioresorbable”. Bioresorbable refers to the ability of being completely metabolized by the human or animal body. This ability is suitable for certain applications, for example when a hemostatic agent is placed in an antrum or other body cavity.

The splint according to the present disclosure absorbs blood by its hydrophilic nature and porous structure of the foam and displays sufficient strength to remain properly positioned during the time of healing of the wound. New tissue may grow into the absorbent foam 104. After a certain period, which may be controlled by proper selection of the phase-separated polymer used for its manufacture, the foam 104 will degrade to mere residue and may eventually be completely metabolized by the body.

The foam 104 is typically capable of absorbing a water volume that is equal to 2-50, 5-40, or alternatively, 15-35 times its own volume. A good water absorption ensures that the hemostatic foam 104 is capable of absorbing blood. Such a good water absorption is generally provided by the porosity of the foam 104, which can be achieved by selecting a suitable polymer, such as the phase-separated polymer described above.

The foam 104 has a tensile strength of 5-100, preferably 10-50 MPa. The foam 104 preferably has a modulus of 5-100, or 10-75 MPa. The foam 104 may also have a strain at break of at least 200%, preferably at least 400-800%. Such properties can be obtained by selecting a suitable polymer, such as the phase-separated polymer described above.

The splint can have any suitable shape, such as a cylinder, a cuboid, a plate, a flake or a cone.

The biodegradable splint 100 further includes a tissue-adhesive polymer for covalently bonding to tissue in the nasal cavity. The tissue-adhesive polymer includes a reactive functional group. A reactive functional group is intended to include any chemical group, functionality or moiety that may react with tissue and form a covalent bond. Cells (and thus the tissue that is formed by cells) typically comprises protein and carbohydrates on the outer surface that may react in a variety of reactions. Upon placement in the ostium, the tissue-adhesive polymer reacts with the tissue and a covalent bond is formed. For instance, amines of proteins may react with activated ester to form amide bonds, or a sulfide may react with another sulfide to form a disulfide bond. It may be appreciated that other bonding types, such as Van-der-Waals interactions, hydrogen bonding, ionic interaction and the like, may also play a role in the overall bonding capacity of the tissue-adhesive polymer. The specific occurrence and strength of each type of bonding generally depends on the type of tissue, the chemical composition of the tissue-adhesive polymer and the structure of the device based thereon.

The tissue-adhesive functional group is appropriately stable in an aqueous environment, but at the same time sufficiently reactive with respect to the tissue. Although the tissue-adhesive functional group may be sensitive to hydrolysis, appropriately stable means that the group remains stable for a period long enough for the tissue-adhesive polymer to react with the tissue. As such, the tissue-adhesive polymer includes a functional group selected from the group of a carboxylic acid, an activated ester, an acid chloride, an anhydride, an aldehyde, p-nitrophenyl carbonate, epoxide, an isocyanate, vinyl sulfone, maleimide, o-pyridyl-disulfide, a thiol or combinations thereof. Activated esters, acid chlorides, anhydrides, aldehydes, vinyl sulfone, maleimide and isocyanates are electrophilic groups that may typically react with an amine or another nucleophile of the tissue. Thiol or o-pyridyl-disulfide may form a disulfide bond with the tissue.

In certain implementations, the reactive functional group includes activated esters. The activated ester may be a thioester, a perfhioroalkyl ester, a pentafluorophenol ester, a N-hydroxysuccinimide (NHS) ester, derivatives thereof, as well as combinations of these. Particularly good results have been obtained with N-hydroxysuccinimide ester, as this ester is stable enough to allow easy handling but also allows a good tissue-bonding. N-hydroxysuccinimide ester or derivatives thereof is therefore most preferred. Examples of derivatives of NHS ester are N-hydroxysulfosuccinimide and salts thereof.

In certain implementations, the tissue-adhesive polymer is based on polyethylene glycol, such as on a multi-arm polyethylene glycol (PEG). In particular, a 4-arm or an 8-arm polyethylene glycol may be used.

Using a multi-arm PEG based tissue-reactive polymer thus allows the use of less of this polymer in the blend to obtain the same tissue-adhesive strength or the use of a similar amount of this polymer in the blend to obtain a higher tissue-adhesive strength when for instance compared to linear PEG-based tissue-adhesive polymers. There is thus a strong synergistic effect between PEG-based tissue-adhesive polymers and the multiplicity of the arms of such polymers.

A multi-arm PEG is typically based on a core comprising multiple anchoring groups where polyethylene glycol arms or groups can be joined. Since the core is typically relatively small compared to the PEG arms, the multi-arm PEG may typically be regarded as star-shaped. However, the core may also comprise a larger component such as a polymer (e.g. polyethylene glycol) and may thus also be of a considerable length and weight. A typically core for a 4-arm PEG may for instance be based on erythritol or pentaerythritol while a typically core for an 8-arm PEG may for instance be based on hexaglycerol. The distal end 110 of each arm is typically functionalized with a spacer that is functionalized with the tissue-adhesive functional group.

The multi-arm PEG may be varied in terms of the composition of the core, arm length, spacer composition, spacer length and composition of the tissue-adhesive functional group. The arms in a multi-arm PEG are typically of about the same molecular weight (and thus size).

The tissue-adhesive polymer typically has a molecular weight of 2,000 to 100,000 g/mol, preferably 10,000 to 80,000 g/mol, more preferably 20,000 to 60,000 g/mol, most preferably about 40,000 g/mol. The tissue-adhesive polymer is typically a complex compound that requires elaborative synthetic efforts and as such is relatively expensive in comparison to the the polymer of the carrier 104. It is therefore preferred to use the minimal amount of tissue-adhesive polymer required for an adequate tissue-adhesiveness of the splint. As such, the ratio of carrier polymer and tissue-adhesive polymer in the material (calculated on the weight) is 1:10 to 10:1, or 1:5 to 5:1, or 1:3 to 3:1.

Although not required, in certain implementations, the tissue-adhesive polymer is used in combination with a buffer agent. The buffer agent having a pH of more than 7, such as in the range of 8 to 10. The presence of the buffer agent may optimize the tissue-adhesive properties of the tissue-adhesive polymer. Without wishing to be bound by theory, it is believed that the buffer agent provides a favorable local (preferably elevated) pH-value under which the rate of the reaction between the tissue with the tissue-adhesive polymer is relatively higher.

The buffer agent is preferably not detrimental to the degradation properties of the biodegradable splint 100. In addition, the buffer agent is preferably biocompatible. Accordingly, the buffer agent is typically selected from the group consisting of phosphates (e.g. Na₂HPO₄), carbonates, acetates, citrates, Good's buffers (preferably those applicable in a pH range of more than 7, more preferably more than 8) such as bicine and the like.

Referring back to the carrier 102, as described above, the carrier 102 may increase the structural rigidity of the foam 104. The carrier 102 may also be referred to a reinforcing sheet or reinforcing structure. As shown in FIGS. 2 to 4, the carrier 102 may extend from a proximate end 108 to a distal end 110. Typically, the carrier 102 has a sheet like configuration. As best shown in FIG. 4, the carrier 102 may define the plurality of voids 106. The voids 106 may have any configuration, such as square, circular, triangular, and the like. The carrier 102 may have a first side 112 and a second side 114 opposite the first side 112. The first side 112 and the second side 114 are separated by the width, which is typically nominal in comparison to the length of the carrier 102 when the carrier 102 has the sheet like configuration. The voids 106 my extend from the first side 112 through the second side 114 of the carrier 102, such that the carrier 102 defines voids 106 extending through and perpendicular to the first and second sides 112, 114. When the voids 106 extend entirely through the carrier 102, the voids 106 may also be referred to as holes. In other implementations, the carrier 102 may include the plurality of voids 106 with the voids 106 terminating within the carrier 102 and not extending completely through the carrier 102. In these implementations, the voids 106 may be referred to as cavities. The foam 104 disposed in the plurality of voids 106 typically couples and secures the foam 104 with the carrier 102. In other words, the plurality of voids 106 assist with anchoring the foam 104 to the carrier 102, such that the stiffness of the carrier 102 is at least partially imparted to the foam 104. The foam 104 may also surround and abut the carrier 102 at the first side 112, the proximate end 108, and the distal end 110. When the foam 104 completely surrounds the carrier 102, the foam 104 also abuts the second side 114 in addition to the first side 112, the proximate end 108, and the distal end 110.

Although not required, the carrier 102 may be formed from a polyester polymer or a polyether polymer. For example, the carrier may be selected from the group of, polylactic acid (PLA), poly (lactic-co-glycolic acid) (PLGA), poly(ε-caprolactone) (PCL), poly-3-hydroxybutyrate (or poly-β-hydroxybutyric acid, PHB) and combinations thereof. Other polymeric materials are also contemplated. Alternatively, the carrier is formed from polyesters, polyethers, polyhydroxyacids, polylactones, polyetheresters, polycarbonates, polydioxanes, polyanhydrides, polyurethanes, polyester(ether)urethanes, polyurethane urea, polyamides, polyesteramides, poly-orthoesters, polyaminoacids, polyphosphonates, polyphosphazenes or combinations thereof.

In a particular implementation, the biodegradable splint 100 may include a filler in addition to the polymer of the carrier 102 and the tissue-adhesive polymer. When included, the filler polymer is typically used to add stiffness to the biodegradable splint 100. The filler can include inorganic bioglass, or polymeric based.

Because the carrier 102 increases the structural rigidity of the biodegradable splint 100 the carrier 102 generally has a flexural modulus that is greater than the flexural modulus of the foam 104. This also results in a difference in degradation profile, with the foam 104 biodegrading at a higher rate than that of the carrier 102. In further comparison, the carrier 102 generally has a significantly lower porosity than the foam 104.

Although not required, in certain implementations the carrier 102 may be produced by 3D printing or additive manufacturing.

Although not required, be biodegradable splint 100 may also include a chitosan hemostatic agent. The term “chitosan hemostatic agent” as used herein refers to chitosan or a salt or derivative thereof. The chitosan hemostatic agent may be chitosan or chitosan lactate. Further examples of suitable chitosan salts are chitosan esters of glutamate, succinate, phtalate or lactate, chitosan derivatives comprising one or more carboxymethyl cellulose groups, carboxymethyl chitosan. Other suitable examples of chitosan derivates are chitosan with quarternary groups (like N-thrimethylene chloride, N-trimethylene ammonium). Also, bioactive excipients such as calcitonin or 5-methylpyrrolidinone can be used. The foam may include the chitosan hemostatic agent in an amount of from 2 to 50 wt. %, based on the total weight of the foam.

As mentioned above, chitosan is a polysaccharide comprising D-glucosamine units (deacetylated units) and N-acetyl-D-glucosamine units (acetylated units). Chitosan can be prepared from chitin by deacetylating at least part of the N-acetyl-D-glucosamine in chitin (poly-N-acetyl-D-glucosamine) by hydrolysis. The ratio of D-glucosamine units and N-acetyl-D-glucosamine units in chitosan is typically expressed as the degree of deacetylation. The degree of deacetylation is defined as the percentage of glucosamine units in chitosan that are not acetylated. This percentage thus corresponds to the molar percentage of deacetylated units present in chitosan.

Chitosan as used in the splint may have a degree of deacetylation of 1-100%, more preferably 60-100%, even more preferably 80-100%. Chitosan having a relatively high degree of deacetylation generally shows a high hemostatic effect. However, pure chitin (0% deacetylation) does not have sufficient hemostatic properties. The above values also apply to chitosan present in chitosan salts, as well as to chitosan derivatives (which have acetylated and deacetylated units just like chitosan itself).

Suitable chitosan salts are those wherein the amine within the chitosan has a positive charge and its corresponding counter anion is dissociated in solution. Accordingly, suitable chitosan salts may be salts consisting of a chitosan cation and a counter anion. For example, the chitosan hemostatic agent may be a salt of chitosan with an organic acid, in particular with a carboxylic acid such as succinic acid, lactic acid or glutamic acid. Chitosan salts may for example be selected from the group consisting of nitrate, phosphate, glutamate, lactate, citrate, acetate and hydrochloride salts of chitosan.

In general, a chitosan derivative is a chitosan molecule wherein one or more of the hydroxyl groups and/or the amine group present in chitosan has been substituted. For example, the one or more hydroxyl groups may be substituted to obtain an ether or ester. The amine group may be substituted to obtain an amino group, although this generally results in a decrease in hemostatic activity. Therefore, the amine groups of chitosan are preferably unsubstituted.

The chitosan hemostatic agent used in the splint preferably comprises or is preferably derived from chitosan originating from animals, plants or shellfish. These sources give similar good results with respect to the hemostatic effects described above. Furthermore, synthetic chitosan may also be used.

The chitosan hemostatic agent used in the splint may have a molecular weight in the range of about 1-1000 kDa. The molecular weight of chitosan is preferably in the range of 10-500 kDa, most preferably 100-250 kDa.

When both the tissue-adhesive polymer and the chitosan hemostatic agent is used, both components are generally dispersed throughout the foam 104. In certain implementations, the chitosan hemostatic agent is also dispersed throughout the carrier 102. Typically, the tissue-adhesive polymer is not dispersed throughout the carrier 102.

The biodegradable splint 100 is intended to support and stabilize the septum following septoplasty, achieved though self-adhesion to the mucosal wall. Benefits include structural support to the septum following surgery while also minimizing the adhesion risk between septum and lateral nasal wall, absorbing excessive fluids and facilitating hemostasis. In certain implementations, these benefits are collectively achieved through the combination of the foam 104, carrier 102, tissue-adhesive polymer, and chitosan. It is envisioned that the equally distributed adhesion will lead to better structural support of the septum. Additionally, having the foam 104 in direct contact with the mucosa will lead to better clinical outcomes as it will facilitate a wound healing environment via reduced scarring and crusting. The biodegradable splint 100 is resorbable as a whole and would not be dependent on a follow up visit for removal but rather would vacate through a combination of resorption and degradation (via hydrolysis).

Most nasal fractures cause significant bleeding and so the biodegradable splint 100 is able to address that as an absorptive, resorbable foam 104 while also providing dedicated stenting capabilities via the combination of the foam 104 and carrier 102 to ensure septum stabilization. Furthermore, the foam 104 portion includes tissue-adhesive polymer, which allows the biodegradable splint 100 to adhere to the septum thereby decreasing suturing requirements and operation time.

In another aspect, a method for producing a biodegradable splint 100 for use in a nasal cavity is provided. The method includes providing the phase-separated polymer including an amorphous segment and a crystalline segment. The method further includes providing the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The method further includes blending the phase-separated polymer with the tissue-adhesive polymer to create a blended mixture. The method further includes providing the carrier 102 extending from a proximate end 108 to a distal end 110 and defining the plurality of voids 106. The method further includes combining the carrier 102 and the blended mixture in a mold. Finally, the method includes forming the foam 104 having a porosity of 80-99% from the blended mixture with the foam 104 disposed within the plurality of voids 106.

In another aspect, a biodegradable splint 100 for a nasal cavity is provided. The biodegradable splint 100 includes the carrier 102 extending from a proximate end 108 to a distal end 110 for increasing the structural rigidity of the biodegradable splint 100. The biodegradable splint 100 includes the foam 104 at least partially surrounding the carrier 102, the foam 104 having a porosity of 80-99% and including a phase-separated polymer including an amorphous segment and a crystalline segment. The biodegradable splint 100 further includes the tissue-adhesive polymer dispersed throughout the foam 104 for covalent bonding, the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The biodegradable splint 100 further includes a chitosan hemostatic agent and a buffer agent for establishing an environment in the nasal cavity to promote covalent bonding between the tissue-adhesive polymer and tissue in the nasal cavity. The overall size and dimensions of the biodegradable splint 100 can also be manipulated by cutting.

The biodegradable splint 100 may further include a drug. The drug may be blended with the phase separated polymer or may be provided as discrete foam layer or as a discrete coating. The drug may be any pharmaceutically active compound. It may be molecularly small compounds or larger compounds such as polysaccharides (e.g. chitosan, glycoproteins, amylopectins, polycarbonates, hyaluronic acids, celluloses and the like).

The drug may be an anti-inflammatory agent, a hemostatic agent, an anti-allergen, an anti-cholinergic agent, an antihistamine, an anti-infective, an anti-platelet, an anti-coagulant, an anti-thrombic agent, an anti-scarring agent, an anti-proliferative agent, a chemotherapeutic agent, an anti-neoplastic agent, a pro-healing agent, decongestant, a vitamin, a hyperosmolar agent, an immunomodulator, an immunosuppressive agent, or combinations thereof. In one example, the drug is a steroidal anti-inflammatory agent.

In another aspect, a method for producing the biodegradable splint 100 for use in a nasal cavity is provided. The method includes providing the phase-separated polymer including an amorphous segment and a crystalline segment. The method further includes providing the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof. The method further includes providing the chitosan hemostatic agent and providing the buffer agent for establishing an environment in the nasal cavity to promote covalent bonding between the tissue-adhesive polymer and a septum of the nasal cavity. The method further includes blending the phase-separated polymer, the chitosan hemostatic agent, the buffer agent, and the tissue-adhesive polymer to create a blended mixture. The method further includes providing the carrier 102 extending from the proximate end 108 to the distal end 110 and combining the carrier 102 and the blended mixture in a mold. The method further includes forming the foam 104 having a porosity of 80-99% from the blended mixture to produce the biodegradable splint 100. The method may further include dissolving the polymer used to form the carrier 102 (i.e., a carrier polymer) with a solvent, casting the dissolved carrier polymer on a substrate, and removing the solvent to form the carrier polymer.

### Clauses for Additional Protection:

I. A biodegradable splint for an ostium, the biodegradable splint comprising: a carrier; a foam at least partially surrounding the carrier and disposed in the plurality of voids, the foam comprising a phase-separated polymer including an amorphous segment and a crystalline segment; a tissue-adhesive polymer for covalent bonding to tissue in the ostium; and optionally, a drug.
II. A biodegradable splint for an ostium, the biodegradable splint comprising: a carrier defining a plurality of voids; a foam at least partially surrounding the carrier and disposed in the plurality of voids; and a tissue-adhesive polymer for covalent bonding to tissue in the ostium.
III. A biodegradable splint for a nasal cavity, the biodegradable splint comprising: a carrier; a foam comprising a phase-separated polymer including an amorphous segment and a crystalline segment; and a tissue-adhesive polymer dispersed throughout the foam; a chitosan hemostatic agent dispersed throughout the foam; and a buffer agent.
IV. A method for producing a biodegradable splint for use in a nasal cavity, the method comprising: providing a phase-separated polymer including an amorphous segment and a crystalline segment; providing a tissue-adhesive polymer; blending the phase-separated polymer with the tissue-adhesive polymer to create a blended mixture; providing a carrier; combining the carrier and the blended mixture in a mold; and forming a foam from the blended mixture.

Several example implementations have been discussed in the foregoing description. However, the examples discussed herein are not intended to be exhaustive or limit the invention to any particular form. The terminology that has been used herein is intended to be in the nature of words of description rather than of limitation. Many modifications and variations are possible in light of the above teachings and the invention may be practiced otherwise than as specifically described.

1. A biodegradable splint for a nasal cavity, the biodegradable splint comprising:
a carrier extending from a proximate end to a distal end for increasing the structural rigidity of the biodegradable splint, the carrier defining a plurality of voids;
a foam at least partially surrounding the carrier and disposed in the plurality of voids, the foam having a porosity of 80-99% and comprising a phase-separated polymer including an amorphous segment and a crystalline segment; and
a tissue-adhesive polymer for covalent bonding to tissue in the nasal cavity, the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof.

2. The biodegradable splint as set forth in claim 1 further comprising a chitosan hemostatic agent.

3. The biodegradable splint as set forth in claim 2 wherein the foam includes the chitosan hemostatic agent in an amount of from 2 to 50 wt. %, based on a total weight of the foam.

4. The biodegradable splint as set forth in claim 2 wherein the chitosan hemostatic agent is a chitosan salt or derivative thereof.

5. The biodegradable splint as set forth in claim 2 wherein the chitosan hemostatic agent and the tissue-adhesive polymer are homogeneously dispersed throughout the foam.

6. The biodegradable splint as set forth in claim 1 further comprising a buffer agent for establishing an environment in the nasal cavity to promote covalent bonding between the tissue-adhesive polymer and tissue within the nasal cavity.

7. The biodegradable splint as set forth in claim 1 wherein the tissue-adhesive polymer includes the activated ester and the activated ester is further defined as an N-hydroxysulfosuccinimide (NHS) ester.

8. The biodegradable splint as set forth in claim 1 wherein the carrier is formed from a polyester polymer selected from the group of, polylactic acid (PLA), poly (lactic-co-glycolic acid) (PLGA), poly(ε-caprolactone) (PCL), poly-3-hydroxybutyrate (or poly-β-hydroxybutyric acid, PHB) and combinations thereof.

9. The biodegradable splint as set forth in claim 2 wherein chitosan hemostatic agent is dispersed throughout the carrier.

10. The biodegradable splint as set forth in claim 1 wherein the carrier has a first side and a second side opposite the first side, and the foam surrounds and abuts the carrier at the first side, the proximate end, and the distal end.

11. The biodegradable splint as set forth in claim 10 wherein the plurality of voids extend from the first side through the second side.

12. A method for producing a biodegradable splint for use in a nasal cavity, the method comprising:
providing a phase-separated polymer including an amorphous segment and a crystalline segment;
providing a tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof;
blending the phase-separated polymer with the tissue-adhesive polymer to create a blended mixture;
providing a carrier extending from a proximate end to a distal end and defining a plurality of voids;
combining the carrier and the blended mixture in a mold; and
forming a foam having a porosity of 80-99% from the blended mixture with the foam disposed within the plurality of voids.

13. (canceled)

14. The method of producing the biodegradable splint as set forth in claim 12 further comprising dissolving a polymer forming the carrier with a solvent, casting the dissolved carrier polymer on a substrate, and removing the solvent to form the carrier.

15. The method of producing the biodegradable splint as set forth in claim 14 further comprising combining a chitosan hemostatic agent with the dissolved carrier polymer.

16. The method of producing the biodegradable splint as set forth in claim 12 wherein the carrier has a first side and a second side opposite the first side, and the foam surrounds and abuts the carrier at the first side, the proximate end, and the distal end; and wherein the plurality of voids extend from the first side through the second side of the carrier.

17. (canceled)

18. The method of producing the biodegradable splint as set forth in claim 12 further comprising combining a chitosan hemostatic agent with the phase-separated polymer and the tissue-adhesive polymer.

19. The method of producing the biodegradable splint as set forth in claim 18 wherein the foam includes the chitosan hemostatic agent in an amount of from 2 to 50 wt. %, based on a total weight of the foam, and the chitosan hemostatic agent is a chitosan salt or derivative thereof.

20. (canceled)

21. (canceled)

22. The method of producing the biodegradable splint as set forth in claim 12 further comprising combining a buffer agent for establishing an environment in the nasal cavity to promote covalent bonding between the tissue-adhesive polymer and tissue within the nasal cavity with the phase-separated polymer and the tissue-adhesive polymer to form the blended mixture.

23. The method of producing the biodegradable splint as set forth in claim 22 wherein the tissue-adhesive polymer includes the activated ester and the activated ester is further defined as an N-hydroxysulfosuccinimide (NHS) ester and wherein wherein the carrier is formed from a polyester polymer selected from the group of, polylactic acid (PLA), poly (lactic-co-glycolic acid) (PLGA), poly(ε-caprolactone) (PCL), poly-3-hydroxybutyrate (or poly-β-hydroxybutyric acid, PHB) and combinations thereof.

24. (canceled)

25. A biodegradable splint for a nasal cavity, the biodegradable splint comprising:
a carrier extending from a proximate end to a distal end for increasing the structural rigidity of the biodegradable splint;
a foam at least partially surrounding the carrier, the foam having a porosity of 80-99% and comprising a phase-separated polymer including an amorphous segment and a crystalline segment; and
a tissue-adhesive polymer dispersed throughout the foam for covalent bonding, the tissue-adhesive polymer including a functional group selected from a carboxylic acid, an activated ester, an acid chloride, an anhydride, an aldehyde, p-nitrophenyl carbonate, an epoxide, an isocyanate, a vinyl sulfone, maleimide, o-pyridyl-disulfide, a thiol, or a combination thereof;
a chitosan hemostatic agent; and
a buffer agent for establishing an environment in the nasal cavity to promote covalent bonding between the tissue-adhesive polymer and tissue in the nasal cavity.

26.-46. (canceled)
